(19) United States
(12) Patent Application Publication
(10) Pub. No.: US 2008/0187592 A1
(43) Pub. Date: Aug. 7, 2008

(54) Cosmetic Composition Comprising A Tensioning Agent And A Hydroxyalkyl Urea

(75) Inventor: Guillaume Cassin, Villebon sur Yvette (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, PARIS (FR)

(21) Appl. No.: 11/720,287

(22) PCT Filed: Nov. 22, 2005
(86) PCT No.: PCT/EP2005/014176

§ 371 Date: Nov. 14, 2007

Related U.S. Application Data
(60) Provisional application No. 60/634,283, filed on Dec. 9, 2004.

(30) Foreign Application Priority Data
Nov. 26, 2004 (FR) 0452768

(51) Int. Cl. A61K 9/14; A61K 33/00; A61Q 19/00; A61K 33/06

(52) U.S. Cl. 424/489; 424/600; 424/684

(57) ABSTRACT

The present invention relates to a cosmetic composition, in particular an anti-wrinkle composition, comprising, in a physiologically acceptable medium suitable for topical application to facial skin: (a) at least one tensioning agent chosen from: plant proteins and hydrolysates thereof, mixed silicates, colloidal particles of inorganic filler and synthetic polymers; and (b) at least one hydroxyalkyl urea of given formula. It also relates to the use of this hydroxyalkyl urea for improving the remanence of the tensioning effect afforded by a tensioning agent, and also to a process for treating wrinkled skin by applying to said skin a composition this hydroxyalkyl urea and a tensioning agent.

The present invention relates to a cosmetic composition, in particular an anti-wrinkle composition, comprising, in a physiologically acceptable medium suitable for topical application to facial skin: (a) at least one tensioning agent chosen from: plant proteins and hydrolysates thereof, mixed silicates, colloidal particles of inorganic filler and synthetic polymers; and (b) at least one hydroxyalkyl urea of given formula.

It also relates to the use of this hydroxyalkyl urea for improving the remamence of the tensioning effect afforded by a tensioning agent, and also to a process for treating wrinkled skin by applying to said skin a composition comprising this hydroxyalkyl urea and a tensioning agent.

The general field of the invention is therefore that of ageing of the skin.

In the course of ageing of the skin, various signs appear, reflected in particular by a change in the structure and functions of the skin. One of these main signs is the appearance of fine lines, and deep wrinkles, the size and the number of which increase with age. The microrelief of the skin becomes less even and exhibits an anisotropic characteristic.

It is common practice to treat these signs of ageing with cosmetic compositions containing active agents capable of combating ageing, such as α-hydroxy acids, β-hydroxy acids, ascorbic acid and retinoids. These active agents act in particular on wrinkles by eliminating the dead cells of the skin and accelerating the process of cell renewal and/or by increasing the synthesis of extracellular matrix components (glycosaminoglycans, collagen, elastin) or preventing degradation thereof. However, these active agents have the drawback of being effective in treating wrinkles only after they have been applied for a certain amount of time, i.e. an amount of time that may range from a few days to several weeks.

Now, current needs are increasingly tending towards the production of compositions for obtaining an immediate effect, leading rapidly to smoothing-out of the wrinkles and/or fine lines and to the disappearance, even temporary, of fatigue marks. Such compositions are compositions comprising tensioning agents. It is specified that the term “tensioning agent” is intended to mean compounds capable of having a tensioning effect, i.e. compounds that can make the skin taut and bring about a reduction in or even the immediate disappearance of wrinkles, fine lines and fatigue marks.

These tensioning agents may in particular be polymers of natural or synthetic origin in an aqueous dispersion, capable of forming a film that causes shrinkage of the stratum corneum, the superficial or horny layer of the epidermis. The cosmetic or dermatological use of such polymer systems for attenuating the effects of ageing of the skin is described in Patent Application WO 98/29091. Other tensioning agents consist of dispersions of inorganic colloidal particles, in particular of silica, as described in Patent Applications FR-A-2 823 113, FR-2 843 024 and FR-2 659 551 or in U.S. Pat. Nos. 3,819,825 and 4,777,041, for example. Other additional tensioning agents are mixed silicates such as those described in Application FR-2 816 315.

However, these tensioning agents sometimes give a sensation of discomfort to certain users, in particular those who have fragile skin. In addition, the tensioning effect that they afford does not last very long, since the film formed on the skin has a tendency to crack as a result of the facial expressions. The reason for this is that these tensioning agents form a relatively rigid and inflexible film on the skin.

The applicant has noted, surprisingly, that the use of specific hydroxyalkyl ureas in combination with a tensioning agent in a cosmetic composition makes it possible to obtain films that have a satisfactory and long-lasting tensioning effect, said films being flexible and deformable from a mechanical point of view.

Thus, according to a first subject, the invention relates to a cosmetic composition comprising, in a physiologically acceptable medium suitable for topical application to facial skin:
(a) at least one tensioning agent chosen from: plant proteins and hydrolysates thereof, mixed silicates, colloidal particles of inorganic filler and synthetic polymers, and
(b) at least one compound chosen from:

hydroxyalkyl ureas corresponding to general formula (I):

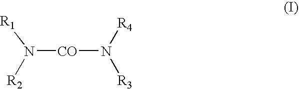

in which R₁, R₂, R₃ and R₄ each independently represent a hydrogen atom, a C₁-C₄ alkyl group or a C₂-C₆ hydroxyalkyl group that may contain from 1 to 5 hydroxyl groups, at least one of the radicals R₁-R₄ representing a hydroxyalkyl group, and

the salts, solvates and isomers thereof.

Among the alkyl groups, mention may be made of methyl, ethyl, n-propyl, isopropyl, n-butyl, isobutyl and tert-butyl groups.

The preferred compounds of formula (I) are those that contain just one hydroxyalkyl group, i.e. those for which R₁ is a hydroxyalkyl group and R₂, R₃ and R₄ represent, independently of one another, a hydrogen atom or a C₁-C₄ alkyl group. The compounds of formula (I) for which R₁ is a hydroxyalkyl group and R₂, R₃ and R₄ each represent a hydrogen atom are more particularly preferred.

Among the hydroxyalkyl groups, those containing a single hydroxyl group, and in particular the hydroxyethyl, hydroxypropyl, hydroxybutyl, hydroxypentyl and hydroxyhexyl groups, are preferred.

As preferred compounds of formula (I), mention may be made of N-(2-hydroxyethyl) urea; N-(2-hydroxypropyl) urea; N-(3-hydroxypropyl) urea; N-(2,3-dihydroxypropyl) urea; N-(2,3,4,5,6-pentahydroxyhexyl) urea; N-methyl-N-(1,3,4,5,6-pentahydroxy-2-hexyl) urea; N-methyl-N′-(1-hydroxy-2-methyl-2-propyl) urea; N-(1-hydroxy-2-methyl-2-propyl) urea; N-(1,3-dihydroxy-2-propyl) urea; N-(trishydroxymethylmethyl) urea; N-ethyl-N′-(2-hydroxyethyl) urea; N,N-bis-(2-hydroxy-ethyl) urea; N,N′-bis-(2-hydroxyethyl) urea; N,N-bis-(2-hydroxypropyl) urea; N,N′-bis-(2-hydroxypropyl) urea; N,N-bis-(2-hydroxyethyl)-N′-propyl urea; N,N-bis-(2-hydroxypropyl)-N′-(2-hydroxyethyl) urea; N-tert-butyl-N′-(2-hydroxyethyl)-N′-(2-hydroxypropyl) urea; N-(1,3-dihydroxy-2-propyl)-N′-(2-hydroxyethyl) urea; N,N-bis-(2-hydroxyethyl)-N′,N′-dimethyl urea; N,N,N′,N′-tetrakis-(2-hydroxyethyl) urea; N′,N′-bis-(2-hydroxyethyl)-N′,N′-bis-(2-hydroxypropyl) urea.

A compound that is particularly preferred for use in the present invention is N-(2-hydroxyethyl) urea, hereinafter referred to as “hydroxyethyl urea”.

The hydroxyalkyl ureas of formula (I) can be prepared as described in application DE-27 03 185. Among these, hydroxyethyl urea is also commercially available, in the form of a mixture at 50% by weight in water, from the company National Starch under the trade name Hydrovance®.

Among the salts, mention may be made of salts of inorganic acids, such as sulphuric acid, hydrochloric acid, hydrobromic acid, hydriodic acid, phosphoric acid or boric acid. Mention may also be made of salts of organic acids, which may contain one or more carboxylic, sulphonic or phosphonic groups. They may be linear, branched or cyclic aliphatic acids or else aromatic acids. These acids may also contain one or more hetero atoms chosen from O and N, for example in the form of hydroxyl groups. Mention may in particular be made of propionic acid, acetic acid, terephthalic acid, citric acid and tartaric acid.

The term “solvate” is intended to mean a stoichiometric mixture of said compound of formula (I) with one or more molecules of water or of organic solvent, such a mixture being derived from the synthesis of the compound of formula (I).

The hydroxyalkyl urea advantageously represents from 0.01 to 20% by weight, and preferably from 1 to 10% by weight, relative to the total weight of the composition.

The other essential constituent of the composition according to the invention is a tensioning agent.

According to the invention, the term “tensioning agent” is intended to mean any agent that produces, at a concentration of 7% in water, a retraction of isolated stratum corneum, measured with an extensometer, of at least 0.9%, and preferably of more than 1.5%, at 30° C. in a relative humidity of 40%.

The principle of the method consists in measuring the length of a test sample of stratum corneum isolated from human skin originating from a surgical operation, before and after treatment with the potential tensioning agent.

To do this, the test sample is placed between the two jaws of the device, one of which is fixed and the other mobile, in an atmosphere of 30° C. and 40% relative humidity. A tensile stress is exerted on the test sample, and the curve of the force (in grams) as a function of the length (in millimetres) is recorded, the length zero corresponding to the contact between the two jaws of the device. The tangent to the curve is then plotted in its linear region. The intersection of this tangent with the x-axis corresponds to the apparent length L₀ of the test sample at zero force. The test sample is consequently slackened and 2 mg/cm² of the composition to be tested (7% solution of the tensioning agent under consideration) are then applied to the stratum corneum. After drying for 15 minutes, the above steps are again carried out in order to determine the length L₁ of the test sample after treatment. The percentage retraction is defined by: % retraction=100×(L₁−L₀)/L₀. In order to characterize a tensioning effect, this percentage must be negative, and the higher the absolute value of the percentage retraction, the greater the tensioning effect.

The tensioning agent is specifically chosen from:
a) plant proteins and hydrolysates thereof;
b) mixed silicates;
c) colloidal particles of inorganic fillers;
d) synthetic polymers;
   and mixtures thereof.

Those skilled in the art will be able to choose, from the chemical categories listed above, the materials corresponding to the tensioning test as described above.

These various categories of tensioning agents will now be described.

a) Plant Proteins and Hydrolysates Thereof

Examples of plant proteins and plant protein hydrolysates that can be used as tensioning agents according to the invention consist of proteins and protein hydrolysates of maize, of rye, of Triticum astivum, of buckwheat, of sesame, of spelt, of pea, of bean, of lentil, of soybean and of lupin.

b) Mixed Silicates

Another class of tensioning agents that can be used according to the invention consists of mixed silicates. This expression is intended to mean any of the silicates of natural or synthetic origin containing at least two different cations chosen from alkali metals (for example, Na, Li or K) or alkaline earth metals (for example, Be, Mg or Ca) and transition metals.

Use is preferably made of phyllosilicates, i.e. silicates having a structure in which the SiO₄ tetrahedra are organized in lamellae between which the metal cations are enclosed.

One of the families of silicates that is particularly preferred as tensioning agents is the laponite family. Laponites are magnesium, lithium, sodium silicates that have a layer structure similar to that of montmorillonites. Laponite is the synthetic form of the natural mineral known as “hectorite”. The laponite sold under the name Laponite XLS or Laponite XLG by the company Rockwood may, for example, be used.

c) Colloidal Particles of Inorganic Filler

As another variant, use may be made, as tensioning agent according to the invention, of colloidal particles of inorganic fillers. The term “colloidal particles” is intended to mean particles in dispersion in an aqueous, aqueous-alcoholic or alcoholic medium, preferably an aqueous medium, having a number-average diameter of between 0.1 and 100 nm, preferably between 3 and 30 nm.

The colloidal particles according to the invention have no thickening property in water, alcohol, oil or any other solvents. At a concentration of greater than or equal to 15% by weight in water, the viscosity of the solution thus obtained is less than 0.05 Pa.s for a shear rate equal to 10 s⁻¹. The measurements are carried out at 25° C. using a Haake RheoStress RS150 rheometer in the cone-plate configuration, the measurements of the measuring cone being: diameter: 60 mm and angle: 2°.

These particles are generally prepared according to a sol-gel process and therefore differ in particular from particles of fumed silica, which agglomerate in water so as to form aggregates larger in size.

Examples of inorganic fillers comprise: silica, cerium oxide, zirconium oxide, alumina, calcium carbonate, barium sulphate, calcium sulphate, zinc oxide and titanium dioxide. An inorganic filler that is particularly preferred is silica. Colloidal silica particles are in particular available in the form of an aqueous dispersion of colloidal silica from the company Catalysts & Chemicals under the trade names Cosmo S-40 and Cosmo S-50.

As a variant, they may be mixed colloidal particles, comprising at least two different types of metal oxides and consisting, for example, of titanium dioxide coated with one or more substances, such as silica-coated titanium dioxide.

A specific example of colloidal particles of inorganic filler may consist of colloidal particles of silica-alumina composite. The term “silica-alumina composite” is intended to mean silica particles in which the aluminium atoms have been partly substituted with silica atoms.

At a pH of 7, these colloidal particles of silica-alumina composite have a zeta potential of less than −20 mV, and preferably less than −25 mV. The measurements are carried out at 25° C. using a Coulter Scientific Instrument Delsa 440SX device.

As silica-alumina composite colloidal particles that can be used in the compositions according to the invention, mention may, for example, be made of those sold by the company Grace under the names Ludox AM, Ludox AM X 6021, Ludox HSA and Ludox TMA.

d) Synthetic Polymers

The synthetic polymers that can be used as a tensioning agent can be chosen from:
polyurethane polymers and copolymers;
acrylic polymers and copolymers;
grafted silicone polymers;
water-soluble or water-dispersible polymers comprising water-soluble or water-dispersible units and units with an LCST.

The polyurethane copolymers, the acrylic copolymers and the other synthetic polymers according to the invention may in particular be chosen from polycondensates, hybrid polymers and interpenetrating polymer networks (IPNs).

For the purposes of the present invention, the term “interpenetrating polymer network” is intended to mean a blend of two intermeshed polymers, obtained by simultaneous polymerization and/or crosslinking of two types of monomer, the blend obtained having a single glass transition temperature.

Examples of IPNs that are suitable for use in the present invention, and also the process for preparing them, are described in patents U.S. Pat. No. 6,139,322 and U.S. Pat. No. 6,465,001, for example.

Preferably, the IPN according to the invention comprises at least one polyacrylic polymer, and it more preferably also comprises at least one polyurethane or a copolymer of vinylidene fluoride and of hexafluoropropylene.

According to a preferred embodiment, the IPN according to the invention comprises a polyurethane polymer and a polyacrylic polymer. Such IPNs are in particular those of the Hybridur series that are commercially available from the company Air Products.

An IPN that is particularly preferred is in the form of an aqueous dispersion of particles with a weight-average size of between 90 and 110 nm and a number-average size of approximately 80 nm. This IPN preferably has a glass transition temperature, Tg, that ranges from approximately −60° C. to +100° C. An IPN of this type is in particular sold by the company Air Products under the trade name Hybridur X-01602. Another IPN that is suitable for use in the present invention has the reference Hybridur X18693-21.

Other IPNs that are suitable for use in the present invention include IPNs consisting of a blend of a polyurethane with a copolymer of vinylidene fluoride and of hexafluoropropylene. These IPNs may in particular be prepared as described in patent U.S. Pat. No. 5,349,003. As a variant, they are commercially available in the form of a colloidal dispersion in water, in a ratio of the fluoro copolymer to the acrylic polymer of between 70:30 and 75:25, under the trade names Kynar RC-10,147 and Kynar RC-10,151, from the company Atofina.

Examples of grafted silicone polymers are indicated in application EP-1 038 519, which is incorporated herein by way of reference. A preferred example of a grafted silicone polymer is polysilicone-8 (INCI name), which is a polydimethylsiloxane onto which are grafted, via a linking chain of thiopropylene type, mixed polymer units of the poly(meth)acrylic acid type and of the poly(alkyl (meth)acrylate) type. A polymer of this type is in particular available under the trade name VS 80 from the company 3M. It is a copolymer of polydimethylsiloxane containing propylthio groups, of methyl acrylate, of methyl methacrylate and of methacrylic acid.

The abovementioned synthetic polymers may be in the form of latex. As a suitable latex that may be used according to the invention as a tensioning agent, mention may in particular be made of polyester-polyurethane and polyether-polyurethane dispersions, such as those sold by the company Noveon under the trade names Avalure UR 405, UR 410 and UR 450.

Finally, synthetic polymers that are particularly suitable may be water-soluble or water-dispersible polymers comprising water-soluble or water-dispersible units and comprising units with an LCST, said units with an LCST having in particular a demixing temperature in water of 5 to 40° C. at a concentration by mass of 1%. This type of polymer is more fully described in patent application FR 2 819 429.

According to a preferred embodiment of the invention, the synthetic polymer, when it is present, is different from an acrylic acid/isobutyl methacrylate/ethyl methacrylate/n-tert-octylacrylamide copolymer such as the polymer sold under the trade name Dermacryl LT by the company National Starch.

The tensioning agent may be included in the composition according to the invention in an amount ranging from 0.01 to 20% by weight of active material, preferably from 1% to 10% by weight of active material, relative to the total weight of the composition. The term “active material” is intended to exclude the medium in which the tensioning agent is optionally solubilized or in dispersion in its commercial form, for example in the case of dispersions of colloidal particles.

The composition according to the invention comprises a physiologically acceptable medium, i.e. a medium compatible with facial skin. It is preferably a cosmetically acceptable medium, i.e. a medium with a pleasant colour, odour and feel, and which does not generate any unacceptable discomfort (stinging, tightness, redness), that may turn the consumer away from using this composition.

The composition according to the invention may be in any of the pharmaceutical forms conventionally used for topical application, and in particular in the form of dispersions of the aqueous lotion or gel type, of emulsions with a liquid or semi-liquid consistency of the milk type, obtained by dispersion of a fatty phase in an aqueous phase (O/W) or vice versa (W/O), or of suspensions or emulsions with a soft, semi-solid or solid consistency of the cream or gel type, or else of multiple emulsions (W/O/W or O/W/O). These compositions are prepared according to the usual methods.

According to a preferred embodiment of the invention, the composition is in the form of an emulsion, and more particularly of an oil-in-water (O/W) emulsion.

This composition may also contain various adjuvants commonly used in the cosmetics field, such as emulsifiers; fillers; preserving agents; sequestering agents; fragrances; thickeners and/or gelling agents.

Of course, those skilled in the art will take care to select this or these optional additional compound(s) and/or the amount thereof in such a way that the advantageous properties of the composition according to the invention are not, or are not substantially, impaired by the envisaged addition.

The composition according to the invention may also contain anti-ageing active agents having an effect complementary to the polymers defined above, such as at least one compound chosen from agents for stimulating collagen and/or elastin synthesis or for preventing degradation thereof, anti-glycation agents, dermo-decontracting agents or muscle-relaxing agents, and mixtures thereof.

A subject of the present invention is also the use of a hydroxyalkyl urea as defined above, for improving the remanence of the tensioning effect afforded by a tensioning agent.

Finally, a subject of the present invention is a process for cosmetically treating wrinkled skin, comprising a step consisting in applying to said skin a composition containing a hydroxyalkyl urea as defined above and at least one tensioning agent.

The application is carried out according to the usual techniques, for example by application of creams, gels, sera or lotions to the skin intended to be treated, in particular the skin around the eyes. In the context of this process, the composition may, for example, be a care composition or a makeup composition.

According to another aspect, the invention also relates to a cosmetic assembly comprising:
i) a container delimiting at least one compartment, said container being closed by means of a closing member; and
ii) a composition as defined above and placed inside said compartment.

The container may be in any appropriate form. It may in particular be in the form of a bottle, a tube, a jar, a case, a box, a sachet or a carton.

The closing member may be in the form of a removable stopper, a lid, a cap, a tear-off strip or a capsule, in particular of the type comprising a body attached to the container and a cover cap articulated on the body. It may also be in the form of a member for selectively closing the container, in particular a pump, a valve or a flap valve.

The product may be contained directly in the container, or indirectly. By way of example, the product may be placed on an impregnated support, in particular in the form of a wipe or of a pad, placed (individually or in plurality) in a box or a sachet. Such a support incorporating the product is described, for example, in application WO 01/03538.

The closing member may be coupled to the container by screwing. Alternatively, the coupling between the closing member and the container is done other than by screwing, in particular via a bayonet mechanism, by click-fastening, gripping, welding, bonding or by magnetic attraction. The term “click-fastening” is intended to mean in particular any system involving the crossing of a bead or cord of material by elastic deformation of a portion, in particular of the closing member, followed by return to the elastically unconstrained position of said portion after the crossing of the bead or cord.

The container may be at least partially made of thermoplastic material. By way of examples of thermoplastic materials, mention may be made of polypropylene or polyethylene.

Alternatively, the container is made of non-thermoplastic material, in particular glass or metal (or alloy).

The containing may have rigid walls or deformable walls, in particular in the form of a tube or a tubular bottle.

The container may comprise means intended to bring about or facilitate the distribution of the composition. By way of example, the container may have deformable walls so as to cause the composition to exit in response to a positive pressure inside the container, said positive pressure being caused by elastic (or non-elastic) squeezing of the walls of the container.

The invention will now be described with reference to the following examples given by way of non-limiting illustration. In these examples, unless otherwise indicated, the amounts are expressed as percentages by weight.

### EXAMPLES

### Example 1

### Evaluation of the Remanence of the Tensioning Effect

The tensioning effect of a dispersion of colloidal silica has already been evaluated in application FR-2 823 113. The ability of the hydroxyalkyl ureas according to the invention to increase the fissuring strength of the tensioning film formed by colloidal silica is evaluated here.

### Protocol

Compositions A to C below were prepared in a manner conventional for those skilled in the art.

| | Composition | Composition | Composition |
|---|---|---|---|
| | A | B | C |
| Hydroxyethyl urea (at 50% | — | 6% | — |
| in water)⁽¹⁾ | | | |
| Glycerol | — | — | 3% |
| Dispersion of colloidal | 17.1% | 17.1% | 17.1% |
| silica particles⁽²⁾ | | | |
| Surfactants | 3.5% | 3.5% | 3.5% |
| Cyclomethicone | 10% | 10% | 10% |
| Stearyl alcohol | 1% | 1% | 1% |
| Sequestering agent | 0.05% | 0.05% | 0.05% |
| Thickeners | 0.6% | 0.6% | 0.6% |
| Preserving agents | 0.9% | 0.9% | 0.9% |
| Water | qs 100% | qs 100% | qs 100% |

⁽¹⁾Hydrovance ® from National Starch
⁽²⁾Dispersion at 41% by weight in water of colloidal silica particles having a mean particle diameter of 18 nm (Cosmo S-40 from Catalysts & Chemicals)

The strengthening potential of glycerol and of hydroxyethyl urea was quantified from the measurement of the breaking strength of materials consisting of compositions A to C.

The test consists in applying a compression stress, to breaking, to the material deposited, by means of a film-drawing device, at the surface of a flexible and deformable foam consisting of neoprene 13 mm thick, the amount of material deposited being such that it makes it possible to obtain a film having a thickness of 15 to 30 μm after drying for 24 h. The use of this foam support makes it possible to impose a considerable distortion on the material deposited at the surface, and therefore to quantify its breaking strength. The mechanical compression stress is exerted after drying of the film, by means of a cylindrical punch 1 mm in diameter; the displacement rate of the punch being 0.1 mm/s. The test is carried out using a TA-XT2i texture analyser sold by the company Stable Micro System. A curve of force F (in N) as a function of displacement d (in mm) is thus obtained, from which it is possible to determine the breaking point of the material.

Two parameters are selected in order to quantify the breaking strength of the material:
\textcircled{1} F_{break} (N): breaking strength
\textcircled{2} W_{break} (J/m²): breaking energy: corresponds to the surface area measured under the curve F=f(d)/surface area of the punch

### Results

The results obtained are given in the table below:

| Composition tested | W_{break} (J/M²) |
|---|---|
| Composition A | 19 ± 3 |
| Composition B | 109 ± 4 |
| Composition C | 54 ± 2 |

These results demonstrate the strengthening role of the hydroxyalkyl urea used in the composition according to the invention, in the presence of a tensioning agent such as colloidal silica. In addition, the mechanical properties of the film obtained are clearly superior with hydroxyalkyl urea than with a conventional plasticizer such as glycerol.

1. A composition comprising, in a physiologically acceptable medium suitable for topical application to facial skin:
(a) at least one tensioning agent selected from the group consisting of plant proteins and hydrolysates thereof, mixed silicates, colloidal particles of inorganic filler and synthetic polymers, and
(b) at least one hydroxyalkyl urea selected from the group of hydroxyalkyl ureas corresponding to general formula (I): 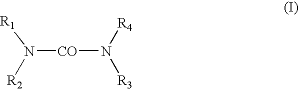
in which R₁, R₂, R₃ and R₄ each independently represent a hydrogen atom, a C₁-C₄ alkyl group or a C₂-C₆ hydroxyalkyl group that may contain from 1 to 5 hydroxyl groups, at least one of the radicals R₁-R₄ representing a hydroxyalkyl group, and
the salts, solvates and isomers thereof.

2. The composition according to claim 1, wherein R₁ is a hydroxyalkyl group and R₂, R₃ and R₄ represent, independently of one another, at least one member selected from the group consisting of a hydrogen atom and a C₁-C₄ alkyl group.

3. The composition according to claim 2, wherein R₁ is a hydroxyalkyl group and R₂, R₃ and R₄ each represent a hydrogen atom.

4. The composition according to claim 1, wherein the compound of formula (I) is selected from the group consisting of N-(2-hydroxyethyl) urea; N-(2-hydroxypropyl) urea; N-(3-hydroxypropyl) urea; N-(2,3-dihydroxypropyl) urea; N-(2,3,4,5,6-pentahydroxyhexyl) urea; N-methyl-N-(1,3,4,5,6-pentahydroxy-2-hexyl) urea; N-methyl-N′-(1-hydroxy-2-methyl-2-propyl) urea; N-(1-hydroxy-2-methyl-2-propyl) urea; N-(1,3-dihydroxy-2-propyl) urea; N-(trishydroxymethylmethyl) urea; N-ethyl-N′-(2-hydroxyethyl) urea; N,N-bis-(2-hydroxy-ethyl) urea; N,N′-bis-(2-hydroxyethyl) urea; N,N-bis-(2-hydroxypropyl) urea; N,N′-bis-(2-hydroxypropyl) urea; N,N-bis-(2-hydroxyethyl)-N′-propyl urea; N,N-bis-(2-hydroxypropyl)-N′-(2-hydroxyethyl) urea; N-tert-butyl-N′-(2-hydroxyethyl)-N′-(2-hydroxypropyl) urea; N-(1,3-dihydroxy-2-propyl)-N′-(2-hydroxyethyl) urea; N,N-bis-(2-hydroxyethyl)-N′,N′-dimethyl urea; N,N,N′,N′-tetrakis-(2-hydroxyethyl) urea; and N′,N′-bis-(2-hydroxyethyl)-N′,N′-bis-(2-hydroxypropyl) urea.

5. The composition according to claim 3 wherein the hydroxyalkyl urea is N-(2-hydroxyethyl) urea.

6. The composition according to claim 1, comprising from 1 to 10% by weight of at least one hydroxyalkyl urea, relative to the total weight of the composition.

7. The composition according to claim 1, wherein the at least one tensioning agent is selected from the group consisting of proteins and protein hydrolysates of maize, of rye, of Triticum aestivum, of buckwheat, of sesame, of spelt, of pea, of bean, of lentil, of soybean and of lupin.

8. The composition according to claim 1, wherein the mixed silicates comprise laponites.

9. The composition according to claim 1, wherein the colloidal particles of inorganic filler are colloidal particles of silica or of silica-alumina composite.

10. The composition according to claim 1, wherein said synthetic polymer is selected from the group consisting of: polyurethane polymers and copolymers, acrylic polymers and copolymers; and grafted silicone polymers.

11. The composition according to claim 10, wherein said synthetic polymer is selected from the group consisting of interpenetrating polymer networks.

12. The composition according to claim 11, wherein said interpenetrating polymer network is in the form of an aqueous dispersion of polyurethane- and polyacrylic-based particles with a weight-average size of between 90 and 110 nm, a number-average size of approximately 80 nm and a glass transition temperature, Tg, that ranges from approximately −60° C. to +100° C.

13. The composition according to claim 10, wherein said grafted silicone polymer is a polydimethylsiloxane onto which is grafted, via a linking chain of thiopropylene type, mixed polymer units of the poly(meth)acrylic acid type and of the poly(alkyl (meth)acrylate) type.

14. The composition according to claim 1, wherein said at least one tensioning agent is included in the composition in an amount ranging from 1% to 10% by weight of active material, relative to the total weight of the composition.

15. (canceled)

16. A process for treating wrinkled skin, comprising applying to said wrinkled skin the composition defined in claim 1.

17. The process according to claim 16 wherein said composition produces, at a concentration of 7% in water, a retraction of the isolated stratum corneum, measured with an extensometer, of more than 0.9%, at 30° C. in a relative humidity of 40%.

18. (canceled)

19. An assembly comprising:
a) a container delimiting at least one compartment, said container being closed by means of a closing member; and
b) a composition placed inside said compartment, the composition being as defined in claim 1.
